(19) United States
(12) Patent Application Publication
(10) Pub. No.: US 2009/0012424 A1
(43) Pub. Date: Jan. 8, 2009

(54) DEVICE FOR REMOVING AND SMEARING CELLS

(75) Inventors: Abdolhamid Huschmand Nia, Gottingen (DE); Reihold Wegener, Gottingen (DE)

Correspondence Address: GREENBLUM & BERNSTEIN, P.L.C., 1950 ROLAND CLARKE PLACE, RESTON, VA, 20191, US

(73) Assignee: GEORG-AUGUST-UNIVERSITAET GOETTINGEN, Goettingen (DE)

(21) Appl. No.: 10/598,530

(22) PCT Filed: Mar. 3, 2005
(86) PCT No.: PCT/DE2005/000370

§ 371 Date: Sep. 1, 2006

(30) Foreign Application Priority Data
Mar. 3, 2004 (DE) 10 2004 010 983.4

(51) Int. Cl. A61B 10/00

(52) U.S. Cl. 600/569

(57) ABSTRACT

A device for removing and smearing cells for a cytological examination with a handle at whose front end a device for the collection of the cells is arranged. The device provides an economical solution for cell collection and a reliable solution for smearing on a microscope slide. The device is embodied as a cone tapering towards the front in which a stabilizing device acting on a longitudinal extension of the cone is arranged.

### FIELD OF THE INVENTION

The invention relates to a device for removing and smearing cells for a cytological examination with a handle at whose front end a device for the collection of the cells is arranged. In particular such a device is suitable for undertaking examinations of the cervix, in particular for taking a smear.

### DISCUSSION OF BACKGROUND INFORMATION

Various methods of undertaking a cell smear are known from prior art, in particular during screening for cervical carcinomas. One method for the collection of the cells comprises the use of a cotton carrier in which cotton is arranged at one end of a strip of wood. Experience has shown that a slight lesion is caused by a cotton carrier, and moreover a deep endocervical cell removal is enabled. A disadvantage of the use of a cotton carrier is the fact that two cotton carriers are required for one smear and that as a rule the smearing pressure is uneven. Too strong a smearing pressure leads to considerable cell- and nucleus degeneration, too low a smearing pressure leads to too small a removal of cells.

Another method or device for the smear removal comprises a Szalay spatula that is embodied with a handle at whose front end a section is equipped for endocervical cell collection. In front of this section, seen from the direction of the handle, a shoulder is mounted that collects the exocervical cells. The Szalay spatula enables a larger cell yield than the cotton carrier as well as a simultaneous removal and smearing of endocervical and ectocervical cells. However, it is disadvantageous that a deep endocervical removal is not always possible and in the case of a tilted cervix, the rear portion of the cervix cannot be covered reliably. Moreover there is a risk of injury to the cervix surface.

The so-called “Cytobrush” is composed of a handle with a brush arranged at the front end, with which the removal of the cells with a high yield is successful even in the case of a narrow cervical canal. The disadvantage associated with it is the danger that vital glandular cells will be torn from their connection, resulting in possible false interpretations. Moreover bleeding can be caused that leads to a limited ability of the removed cells to be evaluated.

Finally there exists the so-called “Zervexbrush,” in which brush elements matched to the cervix contour are arranged at the front end of a handle, with which elements endo- and ectocervical cells can be removed simultaneously. After the removal, the brush head is sent to the laboratory, where the further processing and smearing on a microscope slide takes place. This device is very expensive and is unsuited as a smearing instrument.

From DE 21 35 477A1 a cytological sampler is known in which a foam material cone composed of polyurethane foam is adhered onto a plate fixed on a handle. During the insertion of the collector part, the cone is compressed, which hinders the taking of a sample in the rear cervical canal. Likewise, a deformation of the cone occurs when it is rolled on the microscope slide, which hinders a controlled and uniform application of the cells onto the microscope slide and thus increases the risk of an incorrect finding in the evaluation of the cells, or makes an evaluation impossible. Likewise, an assignment of the cells to a removal point within the cervical canal is not possible. The device is unsuitable and unusable for a cervical smear.

### SUMMARY OF THE INVENTION

The invention provides a device that overcomes the disadvantages of the prior art and provides an economical solution for an exact cell collection and a reliable solution for the smearing onto a microscope slide.

This is achieved according to the invention by a device with the features of claim 1, namely, in that the device is embodied as a cone tapering towards the front, which cone adapts to the cervical canal and the cervical portio and during the cell removal smears both intracervical cells and cells of the portio surface. During the sampling it is necessary to perform a rotary motion, since spatially limited conditions prevail in gynecological use; in particular the possible degrees of freedom for the sampling are limited. The rotatability is necessary due to the minimal space available in the working area, in order to guarantee an optimum cell removal. Due to the arrangement of a stabilization within the device, a precise cell removal is provided together with a simultaneous guarantee of a spatial assignability of the smeared cells on the microscope slide, since no noteworthy length deformation of the removal device occurs either during the cell removal or the smearing.

A further development of the invention provides that the device for the collection of the cells is arranged so that it can rotate relative to the handle. By these means a gentle cell collection can occur at the site to be examined, at the same time through the rolling of the cell collection device on a microscope slide, the cells are laid uniformly on the microscope slide, as a result of which a uniform and non-falsified evaluation of the collected cells is enabled. It is not required that the handle be rotated concomitantly, which leads to blurred smearings if carried out imprecisely.

In addition it is provided that a foam material layer is arranged on the outside of the device for the collection of the cells, on which foam material layer a high yield of cells can attach. In tests, the optimum foam material has been ascertained to be a material of polyurethane foam with a bulk density of 24 kg/m³, a tensile strength of above 110 KPa, an elongation at break of above 120%, a compressive strength of 4.0 KPa at 40% deformation, a deformation by compression of less than 10%, and a pore number of 30 to 38 ppi (pores per inch). Due to the foam material, the danger of an injury to the examined site, usually a tissue adhered to the mucous membrane, is avoided at the same time. The device or the cone is advantageously composed completely of plastic, which effects a greater deformability of the cone, as a result of which the device can adapt itself very well to the cervical canal and the portio. At the same time intracervical cells and cells of the portio surface are smeared. Experimental tests have shown that the optimum dimensions of the foam material cone are a length of approximately 21 mm to 23 mm, a cone base diameter of approximately 14 mm to 16 mm, and a cone angle of approximately 25°. The cone can be embodied slightly curved. With this the best values can be achieved during the cell removal and during the transfer to a microscope slide.

Advantageously the device for the collection of the cells is embodied as a cap that is arranged or fixed on a carrier, so that the cap can be produced simply from a material that collects or carries cells. The cap is produced from polyurethane foam according to the above-named values, or is produced from similar materials with similar values or properties. The carrier as such is produced separately, whereby the cap can be slipped or adhered on the carrier. The cap is preferably slipped on a tip at the front end that also serves to stabilize the foam material.

In order not to hinder the cone or the device from being rolled on a microscope slide and to avoid a possible lesion of the sampling site through a sharp or solid edge at the proximal part of the carrier, the carrier features a base surface whose diameter is smaller than the diameter of the base surface of the cell collection device or smaller than the diameter of the cone base surface. The material that takes up or carries cells, in particular foam material, thereby encloses at least the edge of the cone base surface, on the one hand in order to protect the sampling site from injuries, and on the other hand to guarantee the most complete and uniform cell removal possible. The material or foam material surrounding the edge of the base surface is compressed and limited during the cell removal and due to this evens out the load exerted on the tissue.

Due to the rotatable embodiment, it is also possible to obtain in a simple manner uniform smearing on the microscope slide in two separate strips, with separated areas for intracervical cells and cells of the portio surface.

In particular for purposes of the cell collection, it is provided that a locking mechanism be provided for the torsionally rigid positioning of the cone or of the device on the handle. When the locking mechanism is activated, the removal device can readily be rotated by 360° by these means in order to collect cells. Advantageously the locking mechanism is embodied as a positive engagement element that can be pushed along the longitudinal extension of the handle, which element in the locked position engages in at least one correspondingly embodied recess.

The positive engagement element is embodied, for example, as a flattening, a shoulder, a projection, or a toothing, preferably as a toothing in the sawtooth profile, whereby the positive engagement elements engage in corresponding projections, recesses, or correspondingly embodied toothings. In the case of an embodiment of the positive engagement elements as a sawtooth profile, a locking direction in one direction of rotation can be enabled by means of the position of the teeth; the free rotatability in another direction of rotation can be guaranteed in order to roll the cells on a microscope slide.

In particular with a cell removal in connection with the early detection of cervical carcinoma, a force is applied in the longitudinal extension of the handle during the cell removal. If a spring element loads the positive engagement element in the unlocking direction, a free rotatability of the device or of the cone relative to the handle is guaranteed in principle; only when a force acting in the axial direction is applied do the positive engagement elements engage in the corresponding recesses and lock the device in a torsionally rigid manner, so that during a rotation, the device is moved together with the handle. The force with which the device is pressed against the cervical canal or the portio can also be adjusted via the spring element.

If the device comprises a carrier with a foam material cap arranged thereon, in a further development the carrier is pivoted relative to the handle and features either a positive engagement element or a recess, in order to effect a locking of the carrier relative to the handle.

For reasons of improved handling ability, the handle can feature at least partially an angular cross-section or a round cross-section with a structured surface, so that the handle and the cell collection device can be rotated better and more easily. In connection with the conical and thus rotationally symmetrical device for cell collection, an angular handle, in particular in the embodiment as an angular rod, represents a particularly economical and easy-to-handle solution, with which the cell removal and the smear can be carried out very well. The angular rod enables or facilitates the application of the necessary torque during the rotating cell removal in the cervical canal. Because the removal device is completely surrounded by the cervical canal, the torque required for the rotation is quite high. Since a rotating motion must take place in order to guarantee a good sampling, the angular embodiment of the handle or rod is important.

For reasons of stability and to optimize the results to be achieved in the sampling and during the smearing on a microscope slide, a tip projecting into the foam material, which tip over the carrier or a base- or pressure plate, has proven to be advantageous. The tip stabilizes the foam material in the longitudinal direction during the insertion into the cervix and during the removal procedure. With this a uniform and optimum cell removal is enabled both at the edge of the cervix and in the cervical canal. Likewise, the tip stabilizes the foam material in the transverse direction during the rolling on a microscope slide, as a result of which a uniform and controlled application of the cells is enabled and thus an optimum distribution of the cells on the microscope slide is guaranteed, which leads to a considerable improvement in the evaluation of the cells.

### BRIEF DESCRIPTION OF THE DRAWINGS

Exemplary embodiments of the invention are explained in greater detail below, based on the attached Figures. The Figures show;

FIG. 1 shows a first variant of the invention in perspective view;

FIG. 2 shows a second variant of the invention in the locked and unlocked position;

FIG. 3 shows a third variant of the invention with its components and fully assembled;

FIGS. 4a-4b show enlarged representations of the variants according to FIG. 3 in the locked and unlocked position;

FIG. 5 shows an application example of the device according to FIG. 4;

FIGS. 6a and 6b show component representation and sectional representation of a fourth variant of the invention;

FIGS. 7a and 7b show an assembled device according to FIGS. 6a and 6b in sectional detail view;

FIGS. 8a-8e show a further variant of the device; and

FIGS. 9a-9c show an exemplary embodiment with detail views.

### DETAILED DESCRIPTION OF THE INVENTION

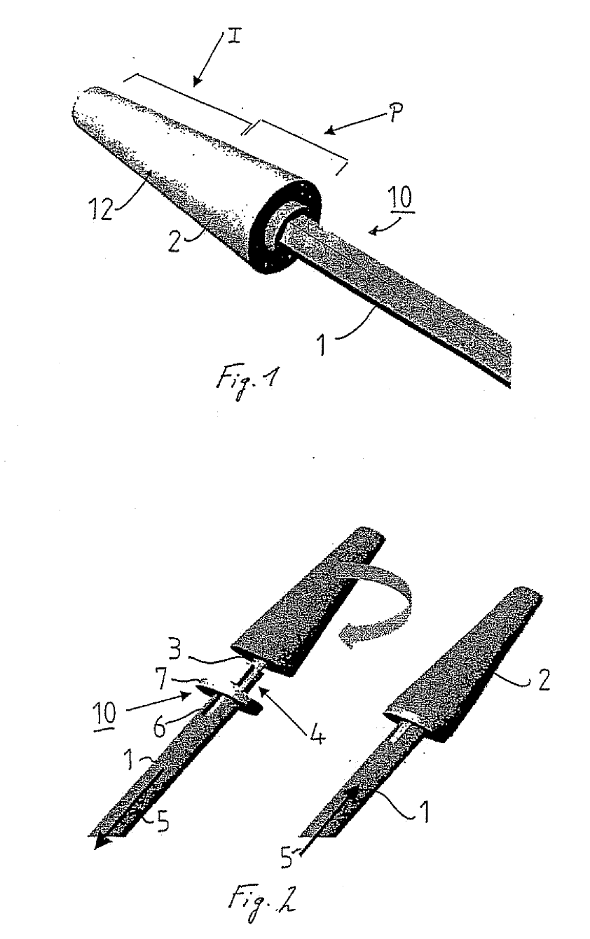

FIG. 1 shows a device 10 for removing and smearing cells for a cytological examination with an angular handle 1, in this case hexagonal in cross-section, and with a device 2 tapering conically towards the front for the collection of cells. The device 2 features on its outer side 12 a layer of foam material to which cells attach during a cell removal, in particular during an early detection for cervical carcinoma. In the front area I the endocervical cells are collected, in the section P of the device 2 facing the handle 1, the cells of the portio surface are taken up. Due to the elastic foam material layer, the cell removal takes place in a manner that is gentle to the tissue and with a higher cell yield than with plastic brushes.

FIG. 2 shows a variant and further development of the device 10 according to the invention, with a handle 1 in which a carrier 3 in the form of a plastic rod is pivoted and can be pushed in the direction of the longitudinal extension 5 of the handle 1. A conical foam material cap 2 is slipped on at the front end of the carrier 3. A recess 6 is embodied in the handle 1, into which recess a corresponding locking mechanism 4 in the form of a shoulder can engage. A pressure plate 7 is arranged at the front end of the handle, in order to support the foam material cap. In the left-hand illustration of FIG. 2 the device 10 is shown in the unlocked position, i.e. the foam material cap 2 can be rotated together with the carrier 3 around the longitudinal extension 5 of the handle 1, as indicated by the arrow. In this unlocked position the cone 2 can be rolled easily on a microscope slide, as a result of which a simple and uniform smearing can be obtained. In the right-hand illustration of FIG. 2 the device 10 is shown in the locked position, i.e., the handle 1 as an outer shell is pushed in the direction of the foam material cap 2. The positive engagement element 4 engages in the recess 6 of the handle 1 with positive engagement and thus by rotating the handle 1 enables a rotation of the foam material cap 2. As a result cells can be collected in the cervix or at another site at which a cell removal is to take place.

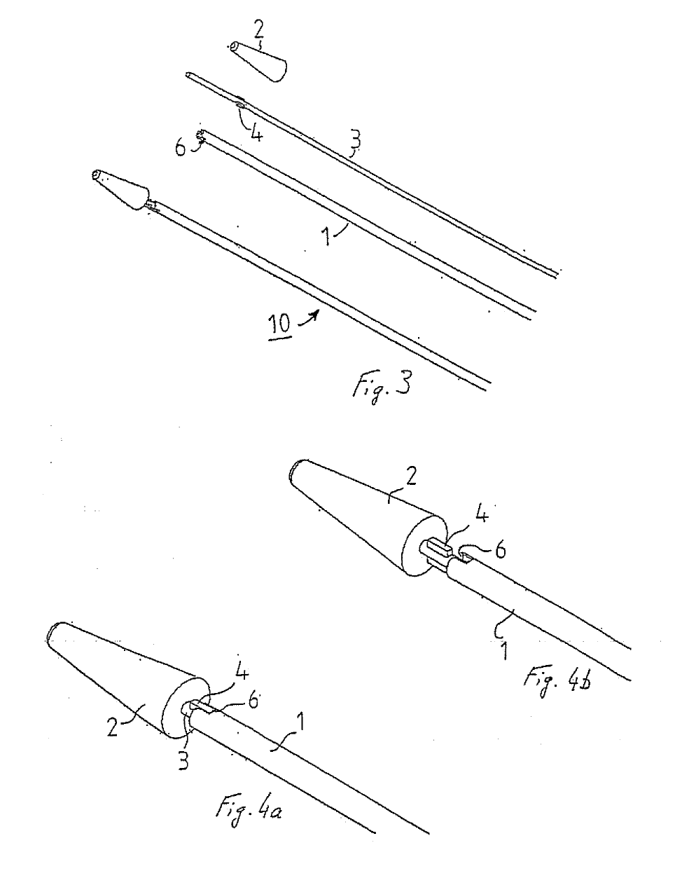

In FIG. 3 another variant of the invention is shown in which no pressure plate 7 is arranged on the handle 1, In the component drawing it can be seen that the carrier 3 comprises a long rod, preferably a plastic rod, that carries two positive engagement elements 4 in the front area. These positive engagement elements 4 are used on the one hand for the torsionally rigid locking of the carrier 3 within the handle 1, on the other hand as a stop for the foam material cap 2. Recesses 6 in the form of slits are routed in the handle 1, in which slits the positive engagement elements 4 of the carrier 3 can engage.

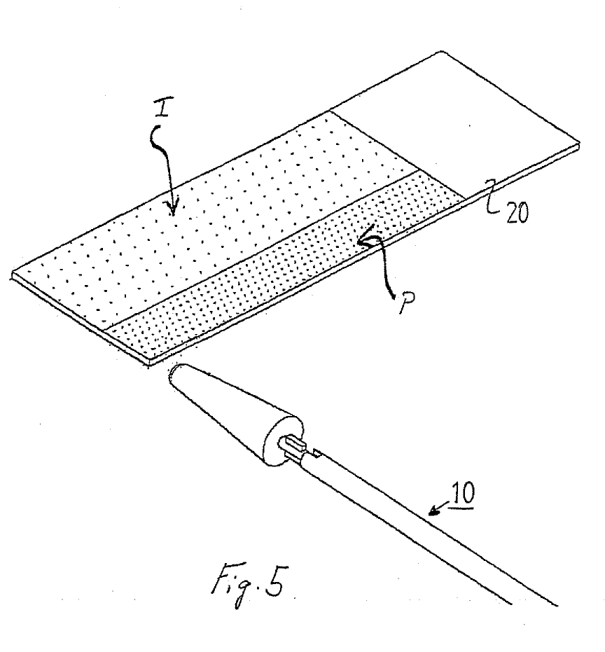

In FIGS. 4a and 4b the variant according to FIG. 3 is shown enlarged. In FIG. 4a the device is shown in the locked position, i.e., the positive engagement elements 4 of the carrier 3 engage in the recesses 6 of the handle 1. In this position a rotating removal of cells is possible. In FIG. 4b the device 10 is shown in the unlocked position, in which a smearing on a microscope slide can take place very precisely and simply.

FIG. 5 indicates how a smearing of the collected cells can take place on a microscope slide 20, namely in that the device 10 is conducted along the surface of the microscope slide 20. This makes it possible for the endocervical cells of the front area I of the foam material cone 2 to be laid on the microscope slide 20 separately from the cells of the portio surface P. In addition to the uniform and gentle as well as fast application of the cells on the microscope slide 20, a precise diagnosis is possible due to the precise assignment of the various source areas.

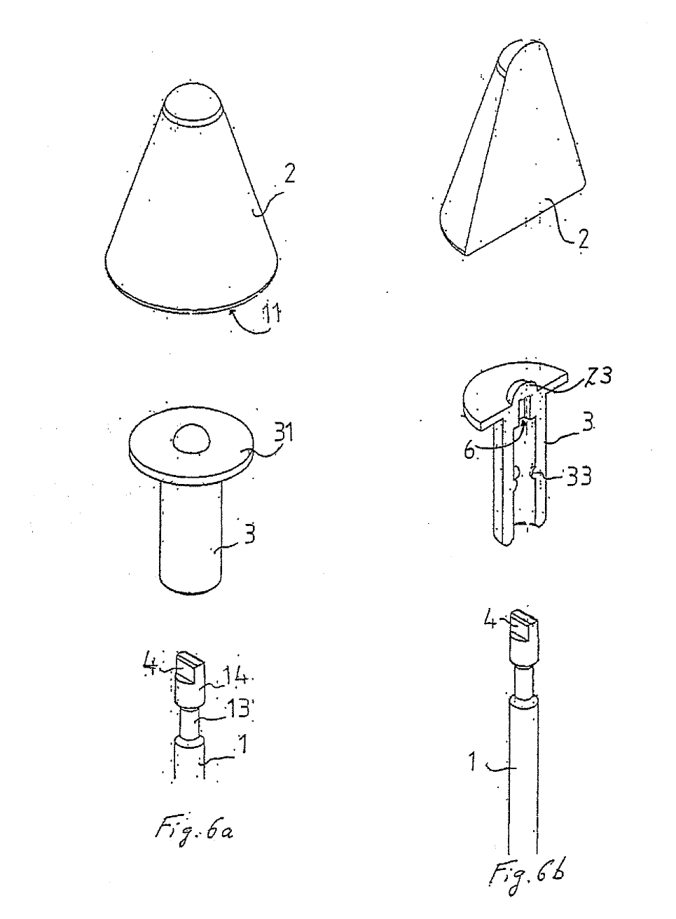

In FIGS. 6a and 6b a variant of the invention is shown in which the handle 1 is embodied essentially round and features a shoulder 13 at the front end, to which shoulder a thickening 14 is adjacent on which positive engagement elements are arranged in the form of shoulders 4. A carrier 3, which features a front end plate 31, is slipped on the handle 1. The foam material cone 2 is placed or adhered on this end plate 31, whereby in this case the foam material cone 2 is composed completely of foam material. Alternatively a foam material layer could be drawn over a core composed of a different material.

In the right-hand FIG. 6b the carrier 3 and the foam material cap 2 is shown in a sectional view. The foam material cap is embodied conically and is composed of foam material; the carrier 3 features a recess 6 for accepting the positive engagement element 4 at the front end of the handle 1. The recess 6 is embodied as a correspondingly embodied slit 6. Within the carrier 3 projections 33 or a peripheral rib are embodied that engage in the shoulder 13 of the handle 1 and thus make it difficult or impossible to pull the carrier 3 off the handle 1.

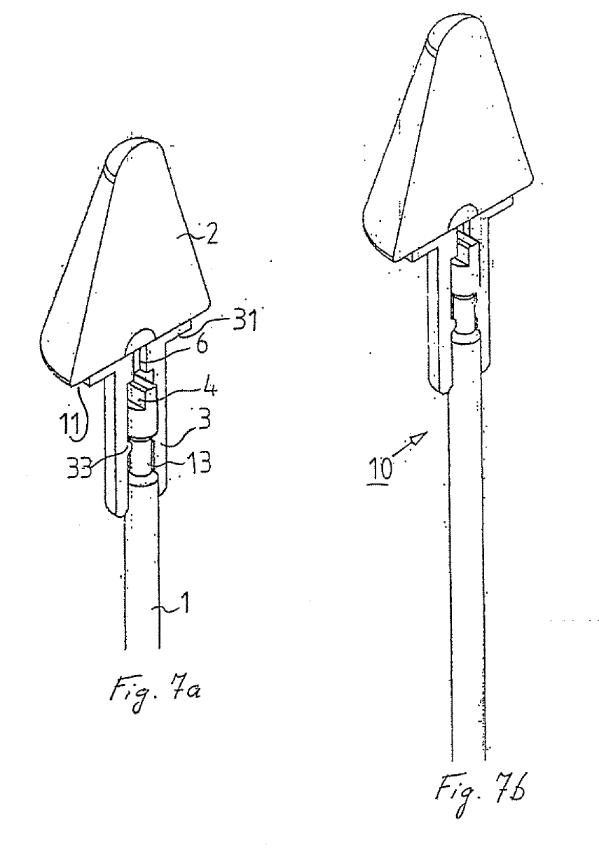

The mode of operation of the device is shown in FIGS. 7a, 7b, in which the components of FIGS. 6a and 6b are shown assembled. In FIG. 7a the foam material cone 2 is mounted, preferably adhered, on the end plate 31, whereby the diameter of the end plate 31 is smaller than the diameter of the base surface 11 of the cone 2. In FIG. 7b the device 10 is shown in an unlocked position, in which both the cone 2 and the carrier 3 can rotate freely around the handle 1, since the positive engagement elements 4 do not engage in the recess 6 of the carrier 3. In order to maintain this condition, a spring can be arranged inside the recess 6 that presses the carrier 3 away from the handle 1. When the cone 2 is inserted into a body cavity, the spring, not shown, is compressed and effects a locking, so that a rotation of the cone 2 can take place through the rotation of the handle 1. In FIG. 7b the device 10 is shown in the locked position. In FIG. 7 it can be seen that a fixing of the carrier 3 to the handle 1 takes place by means of the projection 33 that engages in the shoulder 13.

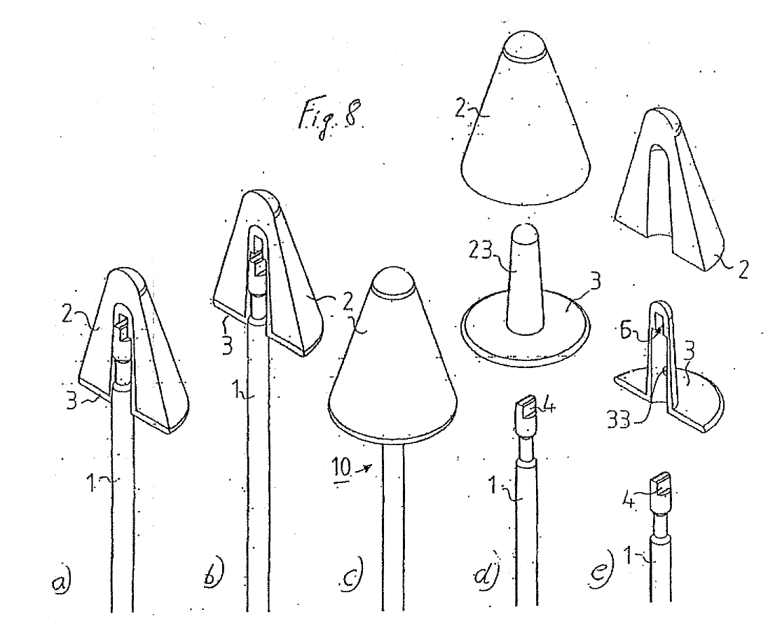

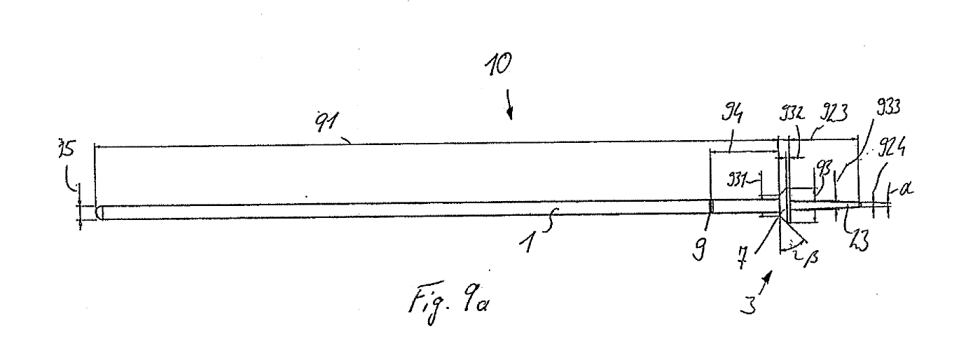

An alternative embodiment of the carrier 3 is shown in FIG. 8, whereby in FIG. 8a a sectional detail view in the locked position and in FIG. 8b a sectional detail view in the unlocked position is shown. The carrier 3 is thereby equipped with a cap-shaped continuation 23, in which both the recess 6 and the projection 33 are arranged. The foam material cone 2 is neither embodied as a solid nor composed completely of foam material, but represents a foam material cover. In other respects the mode of operation corresponds to those of the variant described in FIG. 7.

In order to increase the grip and the rotatability of the device 10, the handle 1 is embodied to be angular, preferably hexagonal or octagonal. Due to the embodiment of a carrier 3 with a carrier plate 31, it is no longer necessary for a catch to be positioned inside the foam material itself, a fact that facilitates the production, since the mechanical elements can be produced by molding, in particular injection molding. As described above, when the cells are smeared, the lock is released and a rolling of the foam material cap 2 on a microscope slide 20 is enabled. Due to the deformability of the foam material cap 2, a cell removal at the portio surface and at the cervical canal can be achieved in one operation. The surface of the foam material- or plastic cap 2 features fine pores that enable a gentle cell removal with a simultaneous high cell yield.

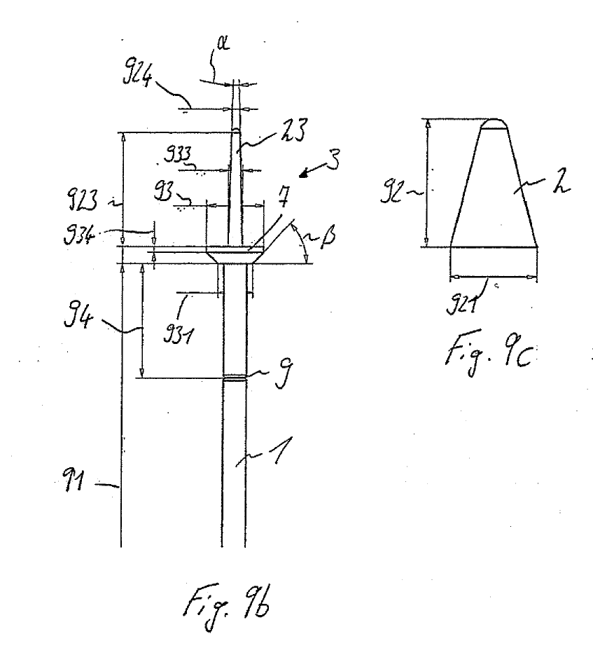

FIG. 9a shows a device 10 embodied as one piece for the removal and smearing of cells, with a handle 1 on which a carrier 3 with a pressure- or base plate 7 is arranged. Not shown is the conical rotationally symmetrical foam material cap that is slipped on a cap-like continuation 23. The continuation 23 serves to stabilize the foam material cap and hinders a compression when the device 10 is being inserted into the cervical canal. This enables a cell removal to be achieved over the entire outer surface of the foam material cap and enables the entire length of the cervical canal to be covered.

Inside the handle 1 a predetermined breaking point 9 is embodied, at which the upper part of the device 10, which serves as carrier 3, can be snapped off and sent packaged to a laboratory. The total length 91 of the handle 1 is approximately ten times the length of the shoulder 94 at which the front part of the device 10 with the continuation 23 and the base plate 7 are arranged. A convenient size for the total length 91 of the handle 1 is approximately 200 mm, the handle diameter 95 is between 3 mm and 7 mm, preferably 4 mm.

In the form of embodiment according to FIGS. 9a and 9b, the base surface 7 on the carrier 3 is embodied conically and after a shoulder 391 whose diameter is 50% larger than the diameter of the handle, widens at an angle β of 45° up to the desired base plate diameter 93, which in an exemplary embodiment features approximately a diameter of 10 mm. An edge 934 enlarges the mounting surface of the foam material cap 2, which is shown in FIG. 9c, and due to the increased surface, reduces the risk of injury during the sampling.

The continuation 23 extending farther from the base surface 7 in the longitudinal extension of the handle 1 features a length 923 that corresponds to the length of the shoulder 931. The diameter 933 at the origin of the continuation 23 is, for example, 2.5 mm, while the tip diameter 924 is smaller and is approximately 1.4 mm to 1.5 mm. The continuation 23 tapers towards the front at an angle α of approximately 3°.

The longitudinal extension 92 of the foam material cap 2 shown in FIG. 9c is greater than the length 923 of the continuation 231 preferably approximately 10% longer. The base diameter 921 of the foam material cap 2 is greater than the diameter 93 of the base surface 7, preferably 50% greater. The foam material cap 2 can also extend beyond the edge 934 in the direction of handle 1, in order to reduce the risk of injury due to the base plate 7. The foam material cap 2 can be slipped or adhered on the continuation 23. By means of the continuation 23, which projects into the foam material cap 2, a function-essential insertion of the foam material cap 2 into the cervical canal can be ensured, without an axial deformation of the foam material cap 2 occurring. The base plate 7 supports the foam material cap 2 and contributes to a stabilization of the foam material cap 2 during the removal.

The handle 1 can be embodied to be angular, while the foam material cap 2 is embodied rotationally symmetrically, in the present case conically.

1-17. (canceled)

18. A device for removing and smearing cells for a cytological examination comprising a handle having a device at a front end thereof for collection of the cells and a stabilizing device acting in a longitudinal extension of the device, the device being embodied as a cap arranged and fixed on a carrier, the carrier including a base surface with a diameter smaller than a diameter of a base surface of the device, the stabilizing device is embodied as a tip projecting into the device, the tip being surrounded on all sides by a foam material, wherein the device is embodied as a cone tapering towards the front and the handle has a predetermined breaking point.

19. The device according to claim 18, wherein the device is arranged to rotate relative to the handle.

20. The device according to claim 18, wherein the device has a foam material layer on its outer side.

21. The device according to claim 18, wherein the device is composed of foam material.

22. The device according to claim 18, wherein the carrier has a base surface with a diameter of 9 mm to 11 mm and a diameter of the device is 12 mm to 18 mm.

23. The device according to claim 18, further comprising a locking mechanism for torsionally rigid positioning of the device on the handle.

24. The device according to claim 23, wherein the locking mechanism is a positive engagement element configured to be pushed along a longitudinal extension of the handle, the positive engagement element, in the locked position, engages in at least one correspondingly embodied recess.

25. The device according to claim 24, wherein the positive engagement element is one of a flattening, a shoulder, a projection, and a toothing in a sawtooth profile.

26. The device according to claim 24, further comprising a spring element loads the positive engagement element in the unlocking direction.

27. The device according to claim 18, wherein the carrier is pivoted relative to the handle and includes one of a positive engagement element and a recess.

28. The device according to claim 18, wherein the handle includes one of an angular cross-section and a round cross-section with a structured surface.

29. The device according to claim 18, wherein the device has a foam material for a cell collection with a pore number of 25 to 40 ppi.

30. The device according to claim 29, wherein the pore number is 32 to 36 ppi,

31. The device according to claim 30, wherein the pore number is 34 ppi.

32. The device according to claim 18, wherein the device has a foam material for the cell collection with a compressive strength of 2 to 6 kPa.

33. The device according to claim 32, wherein the compressive strength is 3 to 5 kPa.

34. The device according to claim 33, wherein the compressive strength is 4 kPa.

35. The device according to claim 18, wherein the device has a cone angle of 20° to 35°.

36. The device according to claim 35, wherein the cone angle is 25° to 30°.

37. The device according to claim 36, wherein the cone angle is 27°.

38. The device according to claim 18, wherein the stabilizing device has a length of 85% to 95% of a length of the device.

39. The device according to claim 38, wherein the stabilizing device has a length of 87% to 93% of the length of the device.

40. The device according to claim 39, wherein the stabilizing device has a length of 90% of the length of the device.

41. The device according to claim 18, wherein the handle has a diameter of 3 mm to 8 mm.

42. The device according to claim 41, wherein the diameter is 4 mm to 7 mm.

43. The device according to claim 42, wherein the diameter is 5 mm to 6 mm.

44. The device according to claim 18, wherein the handle has a total length of 150 mm to 250 mm.

45. The device according to claim 44, wherein the handle has a total length of 180 mm to 220 mm.

46. The device according to claim 45, wherein the handle has a total length of 200 mm.

47. The device according to claim 18, wherein the carrier has a base surface with a diameter of 10 mm,

48. The device according to claim 47, wherein the base surface has a diameter of 15 mm.
